(19) United States
(12) Patent Application Publication
(10) Pub. No.: US 2026/0148831 A1
(43) Pub. Date: May 28, 2026

(54) PET CARE SERVICE MANAGEMENT SYSTEM AND METHOD

(71) Applicant: PetNow LLC, Stone Mountain, GA (US)

(72) Inventor: Adrian Holsendolph, Stone Mountain, GA (US)

(73) Assignee: PetNow LLC, Stone Mountain, GA (US)

(21) Appl. No.: 18/959,981

(22) Filed: Nov. 26, 2024

(51) Int. Cl. G16H 20/60; G06Q 10/1093; G06Q 30/0601

(52) U.S. Cl. CPC G16H 20/60; G06Q 10/1093; G06Q 30/0631

(57) ABSTRACT

A pet care service management system including a transceiver, a memory and a processor is disclosed. The transceiver may receive pet information from a user device, which may include symptom information and diet preference information associated with a pet. The memory may store a mapping between a plurality of pet conditions and a plurality of recommended diets. The processor may estimate a pet condition based on the symptom information, and determine a preliminary diet for the pet by correlating the pet condition with the mapping. The processor may further determine an adjusted diet recipe for the pet based on the preliminary diet and the diet preference information. The processor may additionally transmit information associated with the adjusted diet recipe to the user device.

### FIELD

The present disclosure relates to a pet care service management system and method, and more specifically to a system and method for scheduling a pet care service for a pet, providing a pet symptom checker and recommending an optimal diet for the pet based on pet symptoms and behavior.

### BACKGROUND

A pet owner may regularly require pet care services for the owner's pet. Such services may include emergency services where the pet may require immediate medical attention, e.g., when the pet may be sick or infected. Other types of pet care services may include pet grooming, vaccination, day boarding or overnight boarding services, and/or the like.

A pet owner may face considerable challenges in booking an optimal pet care facility for the pet. A conventional method to book a pet care facility may include calling the pet care facility and speaking with the receptionist about the services provided by the pet care facility, the facility's availability, etc. This is a time consuming and tedious process, which may cause inconvenience to the pet owner. Furthermore, many-a-times, the pet owner may have to call multiple pet care facilities before identifying an optimal and available pet care facility for the pet. Calling multiple pet care facilities may result in loss of precious time, especially if the pet requires immediate medical attention.

Furthermore, there are known instances of the pet owners not knowing what ails their pets and the optimal diets that can be provided to the pets, till they visit a pet care facility/veterinarian to get their pets checked. This may result in a pet (e.g., a sick pet or even a healthy pet) not having optimal diets for a long time, till the pet owner visits the pet care facility/veterinarian.

Therefore, there is a need for a system and method that facilities a user to conveniently book a pet care facility for the user's pet and enables the pet owner to conveniently seek opinion about optimal diets for the pet.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an environment in which techniques and structures for providing the systems and methods disclosed herein may be implemented.

FIG. 2 depicts a first example view of a user device interface displaying a pet care request form in accordance with the present disclosure.

FIG. 3 depicts a second example view of a user device interface displaying a list of available pet care facilities in accordance with the present disclosure.

FIG. 4 depicts a third example view of a user device interface displaying a list of available pet care facilities in accordance with the present disclosure.

FIG. 5 depicts a fourth example view of a user device interface displaying a recommended diet for a pet in accordance with the present disclosure.

FIG. 6 depicts a flow diagram of an example pet care method in accordance with the present disclosure.

### DETAILED DESCRIPTION

### Overview

The present disclosure describes a system and method for scheduling a pet care service for a pet and recommending an optimal diet for the pet based on pet symptoms and behavior. The system may be hosted on a server, and may enable a user to identify an available and optimal pet care facility for the user's pet. The system may communicatively couple with a user device and computing systems associated with a plurality of pet care facilities. The system may receive pet information and user information from the user device, and pet facility information from the computing systems associated with the plurality of pet care facilities.

In an exemplary aspect, the pet information may include symptom information, diet preference information, information associated with one or more required pet care services, and/or a pet type associated with the user's pet. Further, the user information may include information associated with a user's geolocation (e.g., a zip code, or an address), a preferred timeslot for a pet care appointment for obtaining the pet care service for the user's pet, and/or the like. Furthermore, the pet facility information may include information associated with each pet care facility. For example, the pet facility information may include a name or identifier information associated with each pet care facility, information associated with real-time availability at each pet care facility (e.g., for each day of a week, month, etc.), information associated with one or more pet care services provided at each pet care facility, geolocation information associated with each pet care facility, information associated with pet types serviced at each pet care facility, services'pricing, and/or the like.

Responsive to receiving the information described above, the system may correlate the pet information, the user information and the pet facility information to identify one or more optimal pet care facilities for the user's pet. For example, if the pet information indicates that the user's pet is sick and the user information indicates the user's zip code and a preferred appointment time as “immediately” or “within the next 1-2 hours”, the system may identify those pet care facilities that service sick pets and are located at or in proximity to the user's zip code, and are available immediately, based on the pet facility information. The system may further transmit information associated with the identified optimal pet care facilities to the user device, so that the user may select and book an optimal pet care facility for the user's pet.

The system may further recommend optimal diet recipes for the pet before the user arrives at the selected pet care facility at the scheduled appointment time. In this case, the system may first estimate a pet condition based on the symptom information provided by the user. The system may then determine a preliminary diet for the pet based on the pet condition. The system may further determine an adjusted diet recipe for the pet based on the preliminary diet and the diet preference information provided by the user. The system may then transmit information associated with the adjusted diet recipe to the user device. The user may feed the adjusted diet to the pet before the scheduled appointment time, thereby ensuring that the pet's health does not deteriorate further, till the pet is seen by the doctor at the booked pet care facility at the scheduled pet care appointment timeslot.

It is to be noted that the diet recommended by the system is a tool to help alleviate the pet's symptoms from different conditions such as skin allergies, food allergies, kidney disease, joint problems, and/or the like. The recommended diet is not a substitute for the pet's visit to the pet facility and/or the diagnosis of the pet from a veterinarian. As an example, while recommending a diet for the pet on the system's user interface, the user interface may display a message stating “This diet may help your pet's skin abnormalities (redness, itchy, dry, crusting and more). This diet requires a prescription from a veterinarian. Have an exam done on your pet and get the diet best for your pet's needs”.

The system may further enable a pet owner to search for different diets for the pet on the system's user interface. In an exemplary aspect, the pet owner may search for optimal diets for the pet for the pet's skin, heart, kidney, liver, joint, weight care, urinary, and/or the like. The system may further enable the pet owner to search for different supplements for the pet's body parts/parameters described above. The system may further enable the pet owner to search for places that sell homemade pet diet. In further aspects, the system may facilitate the pet owner to prepare an optimal diet for the pet by displaying on the user interface the food items that are rich in Vitamin A, B, C, D, Copper, Iodine, etc.

The present disclosure discloses a pet care service management system and method that facilitates a user to conveniently search for and book an optimal pet care facility for the user's pet. The system quickly and automatically searches for an optimal pet care facility for the user's pet, without requiring the user to call multiple pet care facilities. The system further recommends an optimal diet for the pet based on the pet's condition and behavior, thereby preventing the pet's health from deteriorating further before the pet is seen by the doctor at the pet care facility at the scheduled pet care appointment timeslot.

These and other advantages of the present disclosure are provided in detail herein.

### ILLUSTRATIVE EMBODIMENTS

The disclosure will be described more fully hereinafter with reference to the accompanying drawings, in which example embodiments of the disclosure are shown, and not intended to be limiting.

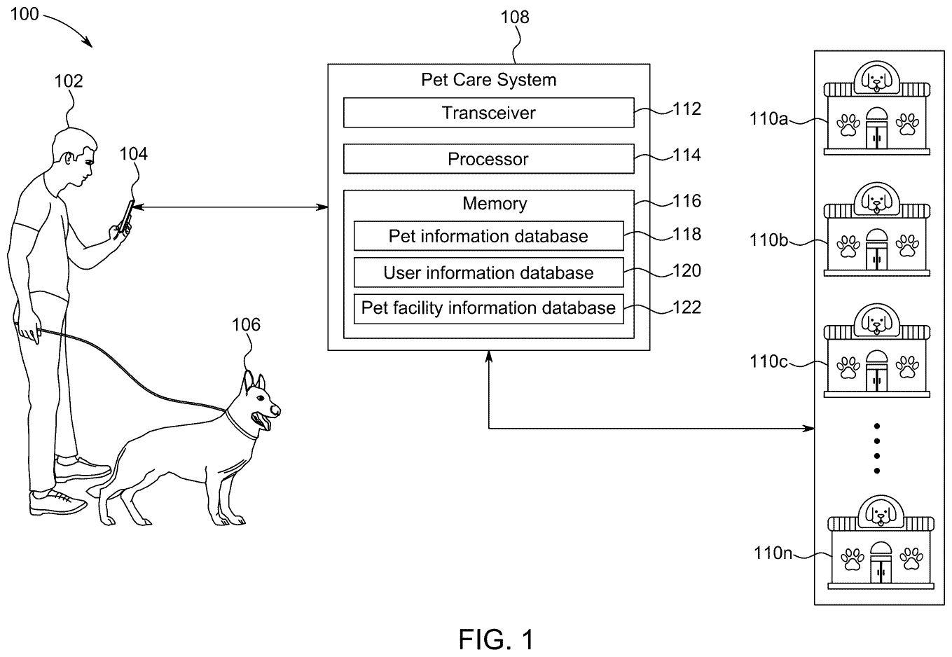

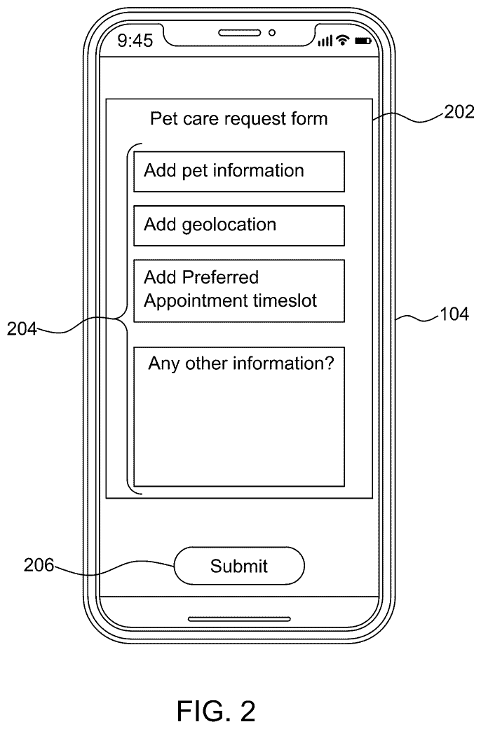

FIG. 1 depicts an environment 100 in which techniques and structures for providing the systems and methods disclosed herein may be implemented. FIG. 1 will be described in conjunction with FIGS. 2, 3, 4 and 5.

The environment 100 may include a pet owner or a user 102 who may be accessing a user device 104. The user device 104 may be, for example, a mobile phone, a desktop computer, a laptop, a tablet, a smartwatch, or any other device with communication capabilities. In some aspects, the user 102 may be accessing the user device 104 to identify an optimal pet care facility for a pet 106 (who may be the user's pet). Although FIG. 1 depicts the pet 106 to be a dog, the present disclosure is not limited to such an aspect. The pet 106 may be any other animal, e.g., a cat, a horse, and/or the like, without departing from the present disclosure scope.

The user 102 may desire to identify an optimal pet care facility for the pet 106 to provide one or more pet care services to the pet 106. Examples of such pet care services may include, but are not limited to, pet treatment (e.g., when the pet 106 may be sick or inflected), grooming (e.g., pedicure, ear cleaning, nail trimming, dental cleaning, etc.), vaccination, day care service, boarding service (e.g., day boarding or overnight boarding), emergency services, and/or the like.

The environment 100 may further include a pet care system 108 (or a pet care management system 108 or “system 108”) that may facilitate the user 102 in identifying one or more optimal pet care facilities for the pet 106. The system 108 may be hosted on a server or a distributed computing system. The system 108 may communicatively couple with the user device 104 (and a plurality of other similar user devices, not shown) and computing systems of a plurality of pet care facilities 110a, 110b, 110c, 110n (or pet facilities, collectively referred to as pet care facilities 110) via a network. Each pet care facility 110 may provide one or more pet care services to pets.

The network, as described above, illustrates an example communication infrastructure in which the connected devices discussed in various embodiments of this disclosure may communicate. The network may be and/or include the Internet, a private network, public network or other configuration that operates using any one or more known communication protocols such as transmission control protocol/Internet protocol (TCP/IP), Bluetooth®, Bluetooth® Low Energy (BLE), Wi-Fi based on the Institute of Electrical and Electronics Engineers (IEEE) standard 802.11, ultra-wideband (UWB), and cellular technologies such as Time Division Multiple Access (TDMA), Code Division Multiple Access (CDMA), High-Speed Packet Access (HSPDA), Long-Term Evolution (LTE), Global System for Mobile Communications (GSM), and Fifth Generation (5G), to name a few examples.

The system 108 may identify one or more optimal pet facilities for the pet 106 based on user and pet's requirements (that the user 102 may provide via the user device 104). The system 108 may additionally recommend an optimal diet (e.g., a nutritious diet/recipe) for the pet 106 based on the pet's condition (e.g., when the pet 106 may be sick or suffering from an ailment). The system 108 may include a plurality of components/units including, but not limited to, a transceiver 112, a processor 114 and a memory 116.

The transceiver 112 may receive data/information/signals from the system 108 components and/or external systems, e.g., the user device 104, the computing systems associated with the pet care facilities 110, etc. via the network described above. For example, the transceiver 112 may receive pet information and user information from the user 102 via the user device 104, and pet facility information associated with the plurality of pet care facilities 110 from the computing systems associated with the pet care facilities 110. In an exemplary aspect, the pet information may include symptom information, diet preference information, information associated with one or more required pet care services, and/or a pet type associated with the pet 106.

The symptom information may include information associated with the symptoms or behavior that the pet 106 may be exhibiting. For example, the user 102 may provide information indicating that the pet 106 is vomiting, having high fever, or experiencing frequent urination as part of the symptom information. In an exemplary aspect, the system 108 may display a “pet symptom checker” on the system user interface (that may be rendered on the user device 104), through which the user 102 may “select” the symptoms that the pet 106 may be exhibiting. For example, the pet symptom checker may display selectable icons displaying text such as “Extreme thirst”, “Always panting”, “Always vomits”, “Excessive urination”, “Always tired”, “Overweight”, “Crusty skin”, “Excessive hair loss and skin redness”, “Always coughs”, “Always hungry”, “Difficulty standing”, “Blood in urine”, and/or the like. The user 102 may select one or more selectable icons on the pet symptom checker to provide the symptom information to the system 108/transceiver 112.

Further, the diet preference information may include information indicating the types of ingredients that the pet 106 may like or dislike. For example, the user 102 may provide information indicating that the pet 106 likes ingredients A, B and C, and dislikes ingredients D, E and F in the pet's diet/food as part of the diet preference information. Furthermore, the information associated with the required pet care services may indicate the pet care services that the pet 106 may require. For example, the user 102 may indicate that the pet 106 requires vaccination or grooming, or an immediate medical attention (e.g., when the pet 106 is sick). Additionally, the pet type may indicate whether the pet 106 is a dog, a cat, a horse, etc., pet's dimensions (e.g., a large-sized dog or a small puppy), pet's age, pet's gender, breed, color, allergies, and/or the like.

The user information may include information associated with a user geolocation (e.g., a zip code, or an address), a preferred timeslot for a pet care appointment for obtaining the pet care service for the pet 106, and/or the like. As an example, the user 102 may indicate in the user information that the user 102 desires to book a pet care service appointment for the pet 106 on Wednesday between 1 PM to 3 PM, as part of the user information.

The pet facility information may include information associated with each pet care facility 110. For example, the pet facility information may include a name or identifier information associated with each pet care facility 110, information associated with real-time availability at each pet care facility 110 (e.g., for each day of a week, month, etc.), information associated with one or more pet care services provided at each pet care facility 110, geolocation information associated with each pet care facility 110, information associated with pet types serviced at each pet care facility 110, and/or the like. For example, the pet facility information may indicate that the pet care facility 110a has availability on Wednesday at 2 PM and services only dogs and cats, the pet care facility 110a address, and information indicating that the pet care facility 110a provides treatment and vaccination services but does not provide grooming or boarding services. Other pet care facilities 110 may have similar associated pet facility information.

Furthermore, the transceiver 112 may transmit data/information/signals to the system 108 components and/or the external systems. For example, the transceiver 112 may transmit to the user device 104 the pet facility information associated with one or more optimal pet care facilities that the system 108 may identify for the pet 106.

The processor 114 may utilize the memory 116 to store programs in code and/or to store data for performing aspects in accordance with the disclosure. The memory 116 may be a non-transitory computer-readable storage medium or memory storing a program code that enables the processor 114 to perform operations in accordance with the present disclosure. The memory 116 may include any one or a combination of volatile memory elements (e.g., dynamic random-access memory (DRAM), synchronous dynamic random-access memory (SDRAM), etc.) and may include any one or more nonvolatile memory elements (e.g., erasable programmable read-only memory (EPROM), flash memory, electronically erasable programmable read-only memory (EEPROM), programmable read-only memory (PROM), etc.).

In some aspects, the memory 116 may include a pet information database 118, a user information database 120 and a pet facility information database 122. The pet information database 118 may store the pet information, the user information database 120 may include the user information and the pet facility information database 122 may store the pet facility information described above. The memory 116 may further store a first data structure or a first mapping between a plurality of pet symptoms and a plurality of pet health conditions (or ailments). For example, the first mapping may indicate that when a pet is experiencing frequent urination, the pet may be having kidney related ailment. As another example, the first mapping may indicate that when a pet is experiencing high fever and frequent vomiting, the pet may be infected.

The memory 116 may further store a second data structure or a second mapping between a plurality of pet health conditions (or “pet conditions”) and a plurality of recommended diets. For example, the second mapping may indicate that when a pet is suffering from infection, the pet should be given a diet “X”; and when the pet is suffering from kidney related ailment, the pet should be given a diet “Y”. The second mapping may further include correlations between different pet types (e.g., pet breed, pet age, etc.) with the recommended diets.

In operation, the user 102 may access an application (“app”) associated with the system 108 on the user device 104 when the user 102 desires to identify an optimal (and available) pet care facility for providing one or more pet care services to the pet 106. Responsive to the user 102 accessing the app, the system 108 may cause the user device 104 to render a pet care request form 202 (as shown in FIG. 2) on a user device screen. The example depiction of the pet care request form 202 in FIG. 2 is provided just for illustrative purpose and should not be construed as limiting. The pet care request form 202 may be of any other type (e.g., in the form of a Question and Answer (Q&A) chat box, a drop down menu, etc.) without departing from the present disclosure scope.

The user 102 may add the pet information, the user information (e.g., the user's geolocation/zip code, the preferred time for pet care appointment, etc.) and/or any other information (e.g., special instructions) in one or more input fields 204 of the pet care request form 202. The examples of the pet information and the user information are described above. Further, as described above, the user 102 may use the pet symptom checker (that may be part of the input fields 204) to provide the symptom information (that may be part of the pet information) to the system 108. Responsive to the user 102 adding the information described above in the input fields 204, the user 102 may click on a submit button 206.

The user device 104 may transmit the information added by the user 102 to the transceiver 112 via the network when the user 102 clicks on the submit button 206. Stated another way, the transceiver 112 may receive the pet information and the user information from the user device 104 when the user 102 adds the information in the input fields 204 and clicks the submit button 206. The transceiver 112 may transmit the received information to the processor 114 and the memory 116 for storage purpose.

Responsive to obtaining the pet information and the user information, the processor 114 may fetch the pet facility information from the pet facility information database 122. The processor 114 may then correlate the pet information, the user information and the pet facility information to identify one or more optimal pet care facilities, from the pet care facilities 110, for the pet 106. For example, if the pet information indicates that the pet 106 is a sick dog and the user information indicates the user's zip code and a preferred appointment time as “immediately” or “within the next 1-2 hours”, the processor 114 may identify those pet care facilities (e.g., the pet care facilities 110a, 110b) that are located at or in proximity to the user's zip code, service sick dogs and are available immediately, based on the pet facility information obtained from the pet facility information database 122.

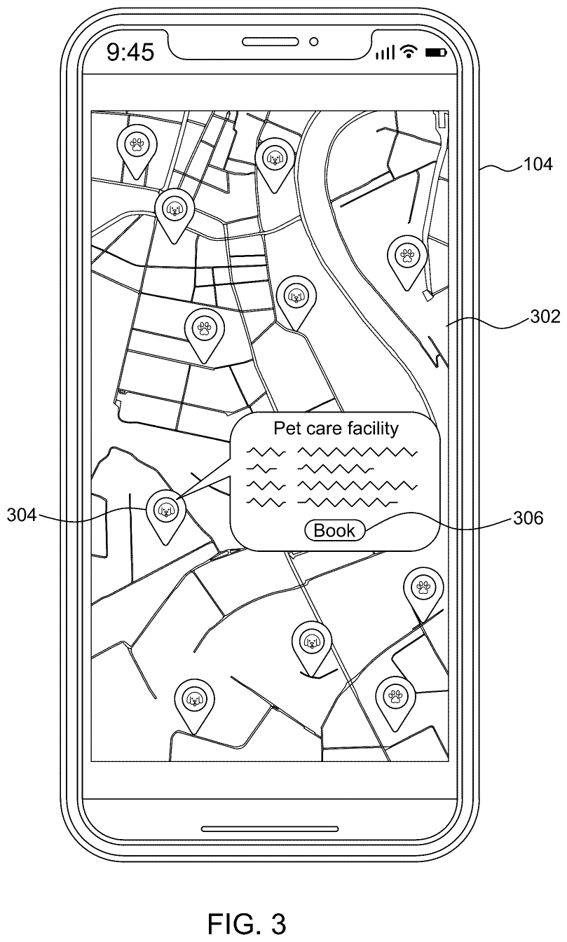

Responsive to identifying the optimal pet care facilities (e.g., the pet care facilities 110a, 110b) as described above, the processor 114 may transmit (e.g., via the transceiver 112) the pet facility information associated with the identified optimal pet care facilities to the user device 104 via the network. In one exemplary aspect, responsive to transmitting the pet facility information, the processor 114 may cause the user device 104 to render the pet facility information associated with the identified optimal pet care facilities on a user device screen in the form of a map 302 (e.g., of a geographical area in and around the user's zip code), as shown in FIG. 3. The map 302 may include location icons 304 that indicate the geolocations of the identified optimal pet care facilities in the geographical area. Further, the map 302 may display the pet facility information associated with a pet care facility when the user 102 hovers on or clicks a location icon associated with the pet care facility. For example, the map 302 may display the address, a distance from the user's geolocation, details of pet care services provided, other users'reviews and feedback, available timeslots, etc. associated with the pet care facility 110a when the user 102 hovers over the location icon associated with the pet care facility 110a.

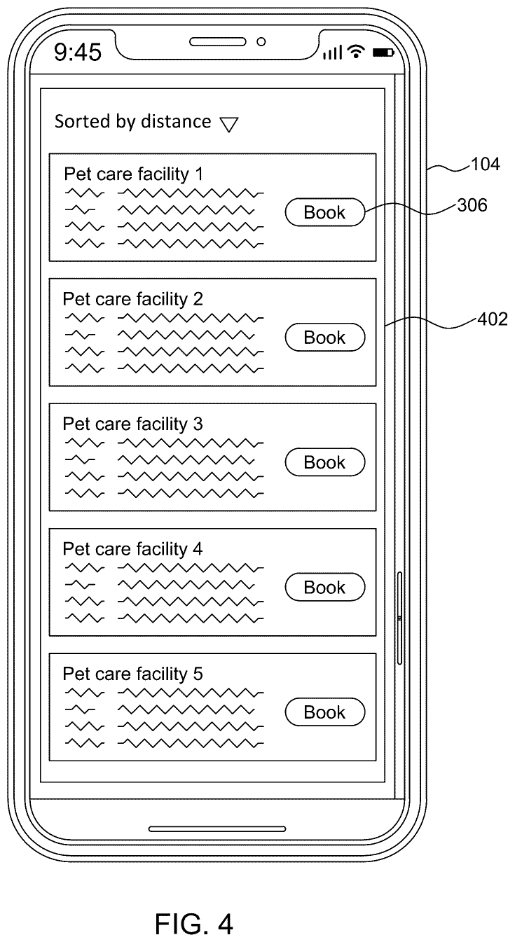

In another exemplary aspect, responsive to transmitting the pet facility information, the processor 114 may cause the user device 104 to render the pet facility information associated with the identified optimal pet care facilities on the user device screen in the form of a list 402 in an ascending order of a distance of each identified optimal pet facility from the user's geolocation, as shown in FIG. 4. For example, the list 402 may display the identified optimal pet facility that is closest to the user's geolocation on the top of the list 402, and the identified optimal pet facility that may be farthest from the user's geolocation on the bottom of the list 402. Similar to the aspect described above, the user 102 may click or hover over the name/identifier of any identified optimal pet facility to view its associated pet facility information.

In additional aspects, the map 302 or the list 402 may enable the user 102 to book (or reserve a booking) at any of the identified optimal pet facility by clicking on a book link 306 on the map 302 or the list 402. For example, the user 102 may click on the book link 306 associated with the pet care facility 110a when the user 102 desires to book a reservation at the pet care facility 110a (e.g., after viewing/reading the pet facility information associated with the pet care facility 110a). The user 102 may additionally input/select the appointment timeslot at the pet care facility 110a when the user 102 clicks on the book link 306.

Responsive to the user 102 clicking on the book link 306, the processor 114 may receive/obtain (via the transceiver 112) a booking request associated with the pet care facility 110a from the user device 104. The processor 114 may then transmit a booking command signal to the computing device associated with the pet care facility 110a to reserve the booking slot for the user 102/pet 106 based on the booking request, and also update the information associated with availability for the pet care facility 110a based on the booking request. For example, if the user 102 has booked the pet care facility 110a for a timeslot of the “next 45 minutes”, the processor 114 may update the availability information for the pet care facility 110a (in the pet facility information database 122 and/or the computing device associated with the pet care facility 110a) to indicate that the pet care facility 110a is unavailable for the next 45 minutes.

In this manner, the system 108 facilities the user 102 in identifying and booking an optimal and available pet care facility for the pet 106 immediately or at any other preferred timeslot, based on the pet's requirements and user preferences (as indicated in the pet information and the user information). Further, the system 108 may provide one or more additional features to the user 102 that may enhance the user's convenience of accessing and using the system 108. Examples of such features are described below.

In one exemplary aspect, during the system operation, the processor 114 may monitor a count of pet care requests associated with each pet care service at different time durations or time slots. Responsive to the monitoring, the processor 114 may perform one or more remedial actions when the count of pet care requests for a specific pet care service increases beyond or greater than a predefined threshold value and when the existing pet care facilities that provide the specific pet care service are not able to fulfil the requests. An example of such a remedial action is described below.

Responsive to determining that the count of pet care requests associated with a specific pet care service (e.g., grooming) at a specific time duration (e.g., for a time slot of 2-3 PM on Wednesday) is greater than the predefined threshold value, the processor 114 may first check whether one or more pet care facilities (from the pet care facilities 110) that provide the specific pet care service are available or unavailable at the specific time duration based on their respective pet facility information. Responsive to determining that such pet care facilities are not available at the specific time duration, the processor 114 may determine an additional pet care facility (from the pet care facilities 110) that is available at the specific time duration and that does not provide the specific pet care service (e.g., grooming) based on its pet facility information.

For example, if the pet care facilities 110a and 110b provide the grooming service, however, these facilities are not available at the specific time duration when the demand for grooming service is high (e.g., greater than the predefined threshold value), the processor 114 may identify the pet care facility 110c that may be available at the specific time duration but may not provide the grooming services. Responsive to identifying the pet care facility 110c that is available at the specific time duration, the processor 114 may transmit a recommendation to the computing device associated with the pet care facility 110c to provide the specific pet care service (i.e., the grooming service) at the specific time duration. The recommendation may be in the form of a request to handle one or more pet grooming requests, so that the system 108 may manage the high demand for grooming services and the pet care facility 110c may also earn additional business when the pet care facility 110c is available (or not attending too many pet patients). In this manner, the system 108 provides benefits to not only the users that seek pet grooming services at their preferred timeslots, but also the pet care facilities by enabling them to earn more business when they have capacity to handle more visitors/pet patients.

In another exemplary aspect, the system 108 may provide recommendations to the user 102 for optimal diets that may be given to the pet 106, e.g., before the user 102 visits the pet care facility for the pet care service at the scheduled appointment/timeslot. It is to be noted that the diet recommended by the system 108 is a tool to help alleviate the pet's symptoms from different conditions such as skin allergies, food allergies, kidney disease, joint problems, and/or the like. The recommended diet is not a substitute for the pet's visit to the pet facility and/or the diagnosis of the pet 106 from a veterinarian.

In this case, the processor 114 may first estimate a pet condition of the pet 106 based on the pet information (specifically the symptom information) provided by the user 102. In some aspects, the processor 114 may use the first mapping between the plurality of pet symptoms and the plurality of pet health conditions described above for estimating the pet condition based on the symptom information provided by the user 102. For example, the processor 114 may estimate that the pet 106 may be infected when the pet 106 may be experiencing high fever and frequent vomiting (as indicated in the symptom information).

In some aspects, the processor 114 may additionally display the estimated pet condition on the user interface associated with the user device 104. For example, the processor 114 may cause the user interface to display “You selected blood in urine and excessive urination as the pet's symptoms. Here are the likely conditions, in no particular order: Bladder stones, Urinary tract infection, Cystitis, Bladder cancer, Kidney disease.”

Responsive to estimating the pet condition, the processor 114 may correlate the pet condition with the second mapping between the plurality of pet health conditions and the plurality of recommended diets described above. The processor 114 may further identify a preliminary recommended diet for the pet 106 based on the correlation of the pet condition with the second mapping. For example, the processor 114 may identify that the diet “X” may be provided to the pet 106 when the pet 106 is infected, based on the second mapping.

The processor 114 may further determine an adjusted diet recipe or an adjusted diet for the pet 106 based on the preliminary diet (e.g., the diet “X”) and the diet preference information that may be part of the pet information provided by the user 102. For example, if the diet preference information associated with the pet 106 indicates that the pet 106 dislikes an ingredient “F” that may be present in the diet “X”, the processor 114 may replace the ingredient “F” with an ingredient “A” (that the pet 106 may like) in the diet “X” to form the adjusted diet recipe. In this case, in some aspects, the ingredients “A” and “F” may be replaceable, with similar effects on the pet's health.

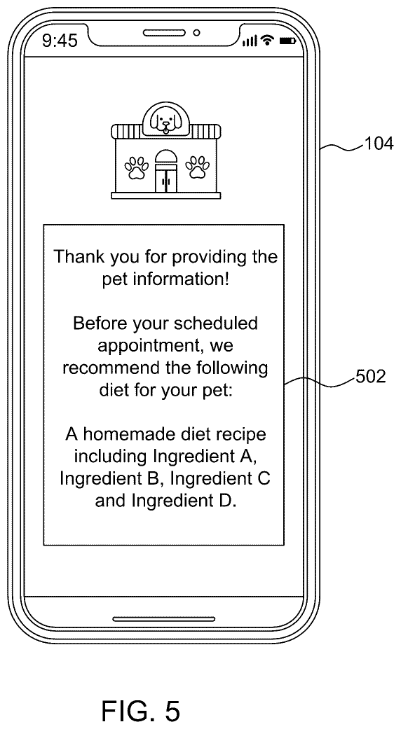

Responsive to determining the adjusted diet recipe, the processor 114 may seek or obtain a doctor confirmation on the adjusted diet recipe (e.g., from a doctor associated with the system 108), and then transmit information associated with the adjusted diet recipe to the user device 104 responsive to obtaining the doctor confirmation. An example recommendation 502 being displayed on the user device 104 is depicted in FIG. 5. The user 102 may view the recommendation 502, and may accordingly feed the pet 106 before the scheduled pet care appointment. The diet recipe included in the recommendation 502 may ensure that the pet's health does not deteriorate further, till the pet 106 is seen by the doctor at the booked pet care facility at the scheduled pet care appointment timeslot.

In some aspects, the processor 114 may determine the preliminary recommended diet for the pet 106 or transmit the information associated with the adjusted diet recipe to the user device 104 after the processor 114 identifies the optimal pet care facilities for the pet 106 based on the pet information, the user information and the pet facility information as described above. In other aspects, the processor 114 may determine the preliminary recommended diet for the pet 106 or transmit the information associated with the adjusted diet recipe to the user device 104 after the processor 114 obtains/receives the booking request from the user device 104. Stated another way, in this case, the processor may determine the preliminary recommended diet for the pet 106 or transmit the information associated with the adjusted diet recipe to the user device 104 after the user 102 books an appointment at a pet care facility on the system 108.

In additional aspects, the system 108 may enable the user 102 to search for different diets for the pet 106 on the system's user interface (that may be rendered on the user device 104). In an exemplary aspect, the user 102 may search for optimal diets for the pet 106 for the pet's skin, heart, kidney, liver, joint, weight care, urinary, and/or the like. The system 108 may further enable the user 102 to search for different supplements for the pet's body parts/parameters described above. The system 108 may further enable the user 102 to search for places that sell homemade pet diet. In further aspects, the system 108 may facilitate the user 102 to prepare an optimal diet for the pet 106 by displaying on the user interface the food items that are rich in Vitamin A, B, C, D, Copper, Iodine, etc.

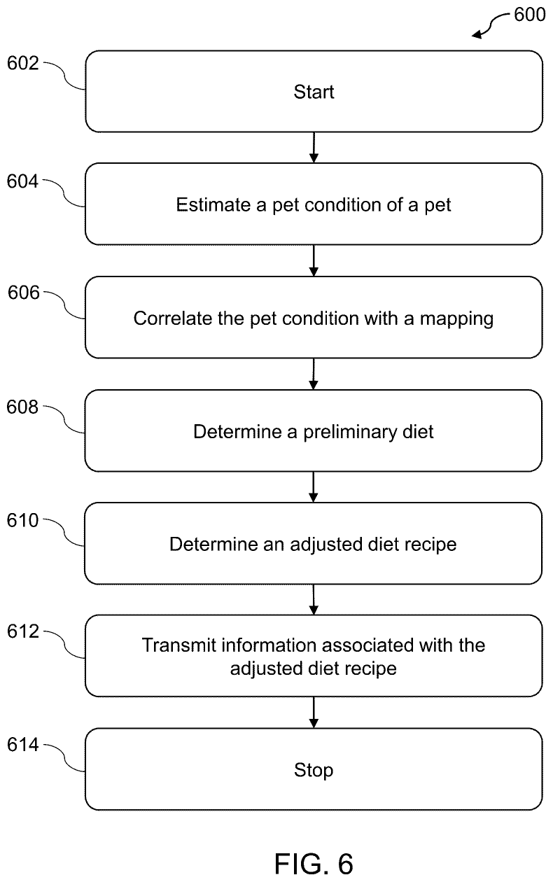

FIG. 6 depicts a flow diagram of an example pet care method 600 in accordance with the present disclosure. FIG. 6 may be described with continued reference to prior figures. The following process is exemplary and not confined to the steps described hereafter. Moreover, alternative embodiments may include more or less steps than are shown or described herein and may include these steps in a different order than the order described in the following example embodiments.

The method 600 starts at step 602. At step 604, the method 600 may include estimating, by the processor 114, the pet condition of the pet 106 based on the symptom information. At step 606, the method 600 may include correlating, by the processor 114, the pet condition with the second mapping described above. At step 608, the method 600 may include determining, by the processor 114, a preliminary diet for the pet 106 based on the correlation.

At step 610, the method 600 may include determining, by the processor 114, the adjusted diet recipe for the pet 106 based on the preliminary diet and the diet preference information, as described above. At step 612, the method 600 may include transmitting, by the processor 114, the information associated with the adjusted diet recipe to the user device 104.

At step 614, the method 600 may stop.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A system comprising:
a transceiver configured to receive a pet information from a user device, wherein the pet information comprises a symptom information and a diet preference information associated with a pet;
a memory configured to store a mapping between a plurality of pet conditions and a plurality of recommended diets; and
a processor configured to:
estimate a pet condition based on the symptom information;
correlate the pet condition with the mapping;
determine a preliminary diet for the pet based on the correlation;
determine an adjusted diet recipe for the pet based on the preliminary diet and the diet preference information; and
transmit an information associated with the adjusted diet recipe to the user device.

2. The system of claim 1, wherein the pet information further comprises an information associated with a pet care service required for the pet and a pet type.

3. The system of claim 2, wherein the pet care service comprises at least one of a pet treatment, a grooming service, a vaccination service, a day care service, or a boarding service.

4. The system of claim 2, wherein the transceiver is further configured to receive a user information from the user device, and wherein the user information comprises a user geolocation and a preferred time for a pet care appointment for obtaining the pet care service.

5. The system of claim 4, wherein the memory is further configured to store a pet facility information associated with a plurality of pet facilities configured to provide a plurality of pet care services, and wherein the pet facility information comprises a name or identifier information associated with each pet facility, an information associated with availability at each pet facility, an information associated with one or more pet care services provided at each pet facility, a geolocation information associated with each pet facility, and an information associated with one or more pet types serviced at each pet facility.

6. The system of claim 5, wherein the processor is further configured to:
correlate the pet information, the user information and the pet facility information;
identify one or more optimal pet facilities, from the plurality of pet facilities, based on the correlation between the pet information, the user information and the pet facility information; and
transmit the pet facility information associated with the one or more optimal pet facilities to the user device.

7. The system of claim 6, wherein the processor is further configured to cause the user device to render the pet facility information associated with the one or more optimal pet facilities on a user device screen in an ascending order of a distance of each optimal pet facility from the user geolocation.

8. The system of claim 6, wherein the processor determines the preliminary diet for the pet after identifying the one or more optimal pet facilities.

9. The system of claim 6, wherein the processor is further configured to:
receive a booking request associated with an optimal pet facility, from the one or more optimal pet facilities, from the user device responsive to transmitting the pet facility information associated with the one or more optimal pet facilities;
transmit a booking command signal to a computing device associated with the optimal pet facility based on the booking request; and
update the information associated with availability for the optimal pet facility based on the booking request.

10. The system of claim 9, wherein the processor determines the preliminary diet for the pet after receiving the booking request.

11. The system of claim 5, wherein the processor is further configured to:
determine that a count of pet care requests associated with a specific pet care service at a specific time duration is greater than a predefined threshold value;
determine that one or more pet facilities providing the specific pet care service are not available at the specific time duration based on the pet facility information, responsive to determining that the count is greater than the predefined threshold value;
determine an additional pet facility, from the plurality of pet facilities, that is available at the specific time duration and that does not provide the specific pet care service based on the pet facility information, responsive to determining that the one or more pet facilities providing the specific pet care service are not available at the specific time duration; and
transmit a recommendation to a computing device associated with the additional pet facility to provide the specific pet care service at the specific time duration, responsive to determining the additional pet facility.

12. The system of claim 1, wherein the processor is further configured to:
obtain a doctor confirmation on the adjusted diet recipe; and
transmit the information associated with the adjusted diet recipe to the user device after obtaining the doctor confirmation.

13. A method comprising:
estimating, by a processor, a pet condition of a pet based on a symptom information, wherein the symptom information is part of a pet information obtained from a user device, and wherein the pet information comprises the symptom information and a diet preference information associated with the pet;
correlating, by the processor, the pet condition with a mapping between a plurality of pet conditions and a plurality of recommended diets;
determining, by the processor, a preliminary diet for the pet based on the correlation;
determining, by the processor, an adjusted diet recipe for the pet based on the preliminary diet and the diet preference information; and
transmitting, by the processor, an information associated with the adjusted diet recipe to the user device.

14. The method of claim 13, wherein the pet information further comprises an information associated with a pet care service required for the pet and a pet type.

15. The method of claim 14, wherein the pet care service comprises at least one of a pet treatment, a grooming service, a vaccination service, a day care service, or a boarding service.

16. The method of claim 14 further comprising:
correlating the pet information, a user information and a pet facility information, wherein the user information comprises a user geolocation and a preferred time for a pet care appointment for obtaining the pet care service, wherein the pet facility information is associated with a plurality of pet facilities configured to provide a plurality of pet care services, and wherein the pet facility information comprises a name or identifier information associated with each pet facility, an information associated with availability at each pet facility, an information associated with one or more pet care services provided at each pet facility, a geolocation information associated with each pet facility, and an information associated with pet types serviced at each pet facility;
identifying one or more optimal pet facilities, from the plurality of pet facilities, based on the correlation between the pet information, the user information and the pet facility information; and
transmitting the pet facility information associated with the one or more optimal pet facilities to the user device.

17. The method of claim 16 further comprising causing the user device to render the pet facility information associated with the one or more optimal pet facilities on a user device screen in an ascending order of a distance of each optimal pet facility from the user geolocation.

18. The method of claim 16 further comprising determining the preliminary diet for the pet after identifying the one or more optimal pet facilities.

19. The method of claim 16 further comprising:
receiving a booking request associated with an optimal pet facility, from the one or more optimal pet facilities, from the user device responsive to transmitting the pet facility information;
transmitting a booking command signal to a computing device associated with the optimal pet facility based on the booking request; and
updating the information associated with availability for the optimal pet facility based on the booking request.

20. A non-transitory computer-readable storage medium having instructions stored thereupon which, when executed by a processor, cause the processor to:
estimate a pet condition of a pet based on a symptom information, wherein the symptom information is part of a pet information obtained from a user device, and wherein the pet information comprises the symptom information and a diet preference information associated with the pet;
correlate the pet condition with a mapping between a plurality of pet conditions and a plurality of recommended diets;
determine a preliminary diet for the pet based on the correlation;
determine an adjusted diet recipe for the pet based on the preliminary diet and the diet preference information; and
transmit an information associated with the adjusted diet recipe to the user device.
